(19) United States
(12) Patent Application Publication
(10) Pub. No.: US 2016/0331360 A1
(43) Pub. Date: Nov. 17, 2016

(54) MEDICAL RETRIEVAL DEVICES AND RELATED METHODS OF USE

(71) Applicant: Boston Scientific Scimed, Inc., Maple Grove, MN (US)

(72) Inventors: Mark HERA, Holden, MA (US); Kenneth ADAMS, Maynard, MA (US); Kimberly DeGRAAF, Holden, MA (US)

(73) Assignee: Boston Scientific Scimed, Inc., Maple Grove, MN (US)

(21) Appl. No.: 15/144,006

(22) Filed: May 2, 2016

Related U.S. Application Data
(60) Provisional application No. 62/160,434, filed on May 12, 2015.

(51) Int. Cl. A61B 17/00; A61B 17/34; A61B 1/07; A61B 1/00; A61B 1/04; A61M 25/00; A61B 1/307

(52) U.S. Cl. CPC A61B 17/00234; A61M 25/0045; A61B 17/3468; A61M 25/005; A61B 1/307; A61B 1/00165; A61B 1/042; A61B 1/07

(57) ABSTRACT

A medical device may include a longitudinally extending body. The longitudinally extending body may define an exterior surface and be configured for into a body of a patient. The medical device may further include a sheath coupled to and surrounding at least a portion of the longitudinally extending body. The sheath may have a selectively expandable mesh of interconnected filaments defining openings between adjacent filaments of the mesh. Further, the mesh may be configured to expand to pass a medical device along the longitudinally extending body, between the sheath and the exterior surface, and through an opening between adjacent filaments of the mesh.

### CROSS-REFERENCE TO RELATED APPLICATIONS

This patent application claims the benefit of priority under 35 U.S.C. §119 to U.S. Provisional Patent Application No. 62/160,434, filed May 12, 2015, which is herein incorporated by reference in its entirety.

### TECHNICAL FIELD

Various aspects of the present disclosure relate generally to devices and related systems and methods. More specifically, the present disclosure relates to devices, systems, and methods for performing a medical procedure on or within a patient.

### BACKGROUND

A variety of medical procedures require the insertion of one or more medical devices and/or systems within a patient's body cavities or passages. Accordingly, such a device and/or system must be sized to be received within said body cavities or passageways. In some arrangements, a medical system may require delivery of a variety of devices to perform a function so as to diagnose and/or treat a patient.

In order to deliver such medical devices (e.g., baskets, snares, laser fibers, etc.) into a subject's body, a medical system may require one or more working channels extending therethrough. Depending on the procedure, however, inclusion of a sufficient number working channels may increase the overall diameter or profile of the medical system. Alternatively, minimizing the size of the working channel(s) of a medical system may limit the usefulness of such working channel(s).

The systems and methods of the current disclosure may rectify some of the deficiencies described above and/or other problems in the art.

### SUMMARY

Examples of the present disclosure relate to, among other things, medical systems, devices, and related methods of use. Each of the examples disclosed herein may include one or more of the features described in connection with any of the other disclosed examples. In one example, a medical device may include a longitudinally extending body. The longitudinally extending body may define an exterior surface and be configured for insertion into a body of a patient. The medical device may further include a sheath coupled to and surrounding at least a portion of the longitudinally extending body. The sheath may comprise a selectively expandable mesh of interconnected filaments defining openings between adjacent filaments of the mesh. Further, the mesh may be configured to expand to pass a medical device along the longitudinally extending body, between the mesh and the exterior surface of the longitudinally extending body, and through an opening between adjacent filaments of the mesh.

Examples of the medical device may include one or more of the following features: the mesh of interconnected filaments may surround an entire circumferential surface the longitudinally extending body; the sheath may further comprise one or more non-mesh portions; the filaments may comprise at least one of flexible wires or polymeric fibers; the mesh of interconnected filaments may comprise filaments of two or more materials; the two or more materials may include at least PTFE and Nitinol; a filler material may extend along at least a portion of the sheath; the filler material may comprise a polymeric material; the filler material may fill in openings between adjacent filaments along the portion of the sheath, the filler material may include a lubricous coating; the longitudinally extending body may further comprise a plurality of lumens; the longitudinally extending body may further define at least one channel extending between a proximal end of the longitudinally extending body and a distal end of the longitudinally extending body; a steering system such that the sheath may be steerable between a first configuration and a second configuration, different than the first configuration; a distal end of the sheath may be fixed to the longitudinally extending body; and the filler material may terminate proximally of a distal-most end of the sheath.

In another example, a method may include delivering a longitudinally extending body into an anatomical opening of a patient. The body may define an exterior surface. At least a portion of the exterior surface may be surrounded by a sheath. The method may further include advancing a medical device along the exterior surface so as to expand the sheath radially outward of the exterior surface. The sheath may comprise a selectively expandable mesh of interconnected filaments defining openings between adjacent filaments of the mesh. Further, the method may include extending the medical device through an opening between adjacent filaments of the mesh.

Examples of the method may include one or more of the following features: actuating the medical device to perform a treatment; the mesh of the interconnected filaments may surround an entire circumferential surface of the body; the sheath may further include one or more non-mesh portions; and the sheath may further include a filler material extending along at least a portion of the sheath.

In another example, a medical device may include a longitudinally extending body defining an exterior surface and configured for into a body of a patient. The medical device may further include a sheath surrounding at least a portion of the longitudinally extending body, and a filler material extending along at least a portion of the sheath. The sheath may comprise a selectively expandable mesh of interconnected filaments defining openings between adjacent filaments of the mesh. Further, the mesh of interconnected filaments may surround an entire circumferential surface of the longitudinally extending body and may be configured to expand to pass a medical device along the longitudinally extending body, between the mesh and the exterior surface, and through an opening between adjacent filaments of the mesh.

Examples of the medical device may include one or more of the following features: the filler material may fill in openings between adjacent filaments along the portion of the mesh; and the mesh of interconnected filaments may comprise filaments of two or more materials, wherein the two or more materials may include at least PTFE and Nitinol.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain aspects of the disclosure.

FIG. 1 depicts a distal portion of an exemplary insertion device according to a first aspect of the disclosure;

FIG. 2 is a perspective view of the insertion device of FIG. 1 including a sheath;

FIGS. 3A-3C depict the delivery of a medical tool via the insertion device of FIG. 1;

FIG. 4A depicts an end view of the insertion device of FIG.1 without a medical device being delivered therethrough;

FIG. 4B depicts an end view of the insertion device of FIG.1 with a medical device being delivered therethrough;

FIG. 5 depicts a distal portion of an exemplary insertion device according to a second aspect of the disclosure;

FIG. 6 depicts a distal portion of an exemplary insertion device according to a third aspect of the disclosure;

FIG. 7 depicts an end view of the insertion device of FIG.6 without a medical device being delivered therethrough; and

FIG. 8 depicts an exemplary insertion device according to a fourth aspect of the disclosure.

### DETAILED DESCRIPTION

Examples of the present disclosure relate to a medical system for diagnosing and/or treating internal areas of a subject's body. The medical system may include an insertion device for facilitating low profile delivery of a medical device.

### Exemplary Embodiments

Reference will now be made in detail to examples of the present disclosure and illustrated in the accompanying drawings. Wherever possible, the same reference numbers will be used throughout the drawings to refer to the same or like parts.

The terms “proximal” and “distal” are used herein to refer to the relative positions of the components of an exemplary medical device. When used herein, “proximal” refers to a position relatively closer to the exterior of the body or closer to a medical professional using the medical device or insertion device. In contrast, “distal” refers to a position relatively further away from the medical professional using the medical device or insertion device, or closer to the interior of the body.

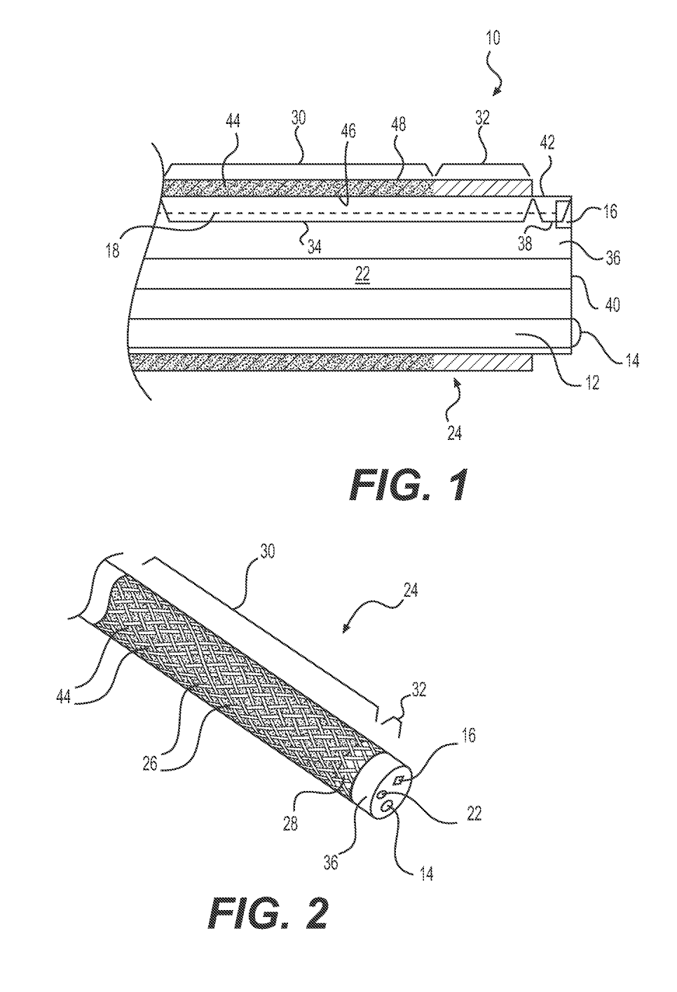

FIG. 1 depicts a portion of an exemplary insertion device 10. Insertion device 10 may include any device configured to allow a user to perform medical diagnoses and/or treatments on a subject. For example, insertion device 10 may include any device configured to allow a user to access and view internal areas of a subject's body. Additionally or alternatively, insertion device 10 may itself be a medical device 20 and/or include any device configured to deliver medical devices 20, such as, for example, guidewires, biopsy forceps, graspers, baskets, snares, probes, scissors, retrieval devices, laser fibers, and/or other tools, into a subject's body. Insertion device 10 may be inserted into one of a variety of body openings, lumens, and/or cavities. For example, the insertion device may be inserted into any portion of a urinary tract, such as a ureter, a gastrointestinal lumen, such as an esophagus, a vascular lumen, and/or an airway.

According to aspects of the present disclosure, insertion device 10 may be a ureteroscope. In some contemplated examples, insertion device 10 may be a sterile, single-use, and disposable ureteroscope. Alternatively, insertion device 10 may be a multiple-use, non-disposable ureteroscope. Other types of devices, however, may be substituted for the ureteroscope, including, as examples, an endoscope, a hysteroscope, a uteroscope, a bronchoscope, a cystoscope, and similar devices. Such devices may be single-use and disposable, or multiple-use and non-disposable.

Insertion device 10 may include a handle (not shown) and a longitudinally-extending insertion body 36 extending distally of the handle. Body 36 may comprise any member sized and/or shaped for insertion within one or more body openings of patient. For example, body 36 may have a circular, ovular, irregular, and/or any other cross-sectional shape appropriate for insertion within anatomical lumens of the subject's body, and/or combinations thereof. An outermost dimension (e.g., diameter) of body 36 may be between about 2.1 mm and 3.2 mm. As used herein, the terms “about,” “substantially,” and “approximately,” may indicate a range of values within +/−5% of a stated value. Body 36 may be comprised of any flexible and/or selectively flexible material, for example to facilitate insertion of body 36 through tortuous anatomy of a patient, as will be described in further detail below. Insertion device 10 may comprise an illumination unit such as light-emitting diodes or fiber optic light guides connected to a proximal source of light (not shown). For example, as shown in FIG. 1, a fiber optic light guide 12 may extend from a proximal source of light (not shown) to a distal end of insertion device 10. The proximal source of light may be included in a proximal control unit (not shown). The proximal control unit may be positioned within the handle of insertion device 10 or a separate control cabinet and may be any appropriate unit configured to execute stored application software and user commands. The proximal control unit may include, for example, a network-enabled image-processing CPU having a memory. Optionally, a distal end of insertion device 10 may include a lens 14 aligned with fiber optic light guide 12. Lens 14 may be configured to focus light emitted by fiber optic light guide 12 onto tissue of a patient to be examined and/or treated. Alternatively or additionally, the illumination unit may include a light emitting diode (LED) or similar illuminating device (not shown) to deliver light to tissue of a patient to be examined and/or treated. In such an example, the LED may be coupled to the proximal control unit via one or more circuits and/or cords (not shown). The cord may be configured to transmit signals to and from the proximal control unit.

Insertion device 10 may also include an imaging unit 16 such as, for example, a miniature video camera and/or image sensor. Exemplary image sensors may include, but are not limited to, CMOS (complementary metal oxide semiconductor) sensors and CCD (charge coupled device) sensors. Imaging unit 16 may be coupled to the proximal control unit via one or more circuits and/or cords 18. Cord 18 may be configured to transmit signals to and from the proximal control unit (not shown). Alternatively, imaging unit 16 may wirelessly communicate with the proximal control unit.

Additionally, insertion device 10 may further define one or more irrigation/aspiration ports (not shown) for communicating fluid therethrough to facilitate removal of organic or inorganic material that may interfere with or obstruct a user's view during a procedure. In some examples, insertion device 10 may be steerable and include an articulable distal tip to facilitate navigation through tortuous anatomy of a patient. Accordingly, one or more steering wires (e.g., Bowden cables) may be provided along insertion device 10 to deflect the distal end of insertion device 10 via manipulation of one or more actuators associated with a proximal end of insertion device 10. Further, insertion device 10 may optionally include one or more working channels 22 configured to deliver one or more medical devices 20 therethrough (not shown in FIG. 1).

Insertion device 10 may include a sheath 24 surrounding at least a portion of body 36. Sheath 24 may comprise a selectively expandable (e.g., stretchable) mesh material, as shown in FIG. 2. At rest (e.g., absent an expanding or stretching force on sheath 24), sheath 24 may have an outermost dimension (e.g., diameter) smaller than an outermost dimension (e.g., diameter) of body 36. Accordingly, when placed over body 36, sheath may fit snuggly along body 36 (FIGS. 1 and 4A). That is, sheath 24 may be in full contact along body 36. Additionally, sheath 24 may be stretchable or expandable away from body 36 of insertion device 10 at any location about body 36. That is, sheath 24 is selectively expandable away from body 36 of insertion device 10 upon the application of an expanding or stretching force on sheath 24.

Sheath 24 may include a plurality of filaments 26 woven together. Filaments 26 may comprise flexible wires and/or polymeric fibers. However, it is understood that the disclosure is not limited to woven sheaths 24, but rather, includes other fabrications such as, for example, knitting, braiding, crocheting, welding, suturing, tying, twisting, or other such methods of producing a sheath with interconnected elements. Exemplary materials for filaments 26 may include, but are not limited to, stainless steel, nylon, polyurethane, urethanes, polyester, Nitinol, PTFE, PET, PEBA, EVA, or any other biocompatible material. In an exemplary aspect, sheath 24 may comprise a co-braid. In other words, sheath 24 may comprise filaments 26 of two or more materials. In one example, sheath 24 may comprise a co-braid of PTFE and Nitinol.

Filaments 26 may be interconnected together to define a plurality of openings 28 between adjacent filaments 26. Filaments 26 may be woven together in any appropriate fashion. For example, filaments 26 may be woven together tightly so as to form a dense mesh having narrow (e.g., small area) openings 28 therebetween. Alternatively, filaments 26 may be loosely woven together to form sparse mesh having wide (e.g., large area) openings 28 therebetween. In another arrangement, a density of the mesh of sheath 24 may vary along the length of sheath 24. For example, a density of mesh of sheath 24 may decrease distally. That is, a main body portion 30 of sheath 24 may be comprised of a relatively high density mesh of filaments 26 defining relatively narrow openings 28, while a distal portion 32 of sheath 24 may be comprised of a relatively low density mesh of filaments 26 defining relatively wide openings 28.

Sheath 24 may extend along body 36 of insertion device 10. For example, sheath 24 may extend from a proximal end of body 36 coupled to a handle (not shown), along portion 34 of body 36, to the distal portion 38 of body 36. For example, sheath 24 may extend distally from the proximal end of body 36 and terminate at distal portion 38 of body 36 prior to a distal-most end 40 of body 36. Alternatively, sheath 24 may extend from the proximal end of the body 36 and terminate at a distal-most end 40 of body 36.

Sheath 24 may be coupled to an exterior surface 42 of body 36 of insertion device 10 via any appropriate means and at any appropriate number of axial locations, such as a proximal end and a distal end of sheath 24. For example, sheath 24 may be welded, glued, or otherwise joined to exterior surface 42 of body 36. In one exemplary aspect, sheath 24 may be tack welded or otherwise coupled to one or more portions of body 36 in any appropriate configuration. In one example, an entire circumferential surface of sheath 24 at a first axial location may be coupled to sheath 24. That is, a 360° weld may be formed between sheath 24 and exterior surface 42 at the first axial location. In other arrangements, a tack weld of less than 360° may be formed between sheath 24 and exterior surface 42. For example, one or more radial arcs of an entire circumferential surface of sheath 24 at a first axial location may be tack welded to exterior surface 42. That is, for example, every other 30° of an entire circumferential surface of sheath 24 at a first axial location may be tack welded to exterior surface 42. In other arrangements, every other 10-180° of an entire circumferential surface of sheath 24 at a first axial location may be tack welded to exterior surface 42. While symmetric coupling (e.g., every other 30°) has been described, it is to be understood that asymmetric coupling (e.g., a first arc of 30° and a space of less or more than 30°) is envisioned and contemplated by this disclosure. In other arrangements, one or more locations along an entire circumferential surface of sheath 24 at a first axial location may be spot welded to exterior surface 42. Such spot welds may be positioned at any desirable location about the circumferential surface of sheath 24 at a first axial location. Additionally, such spot welds may be spaced such that a medical device 20 may be passed between adjacent spot welds.

As shown in FIG. 2, main body portion 30 of sheath 24 may include an axially continuous filler material 44. Alternatively, the sheath 24 may include an axially discontinuous filler material 44 along at least main body portion 30 of sheath 24. Such filler material 44 may be comprised of a lubricious or low coefficient of friction material configured to facilitate passage of one or more medical devices 20 there along, as will be described in further detail below. Accordingly, the filler material 44 may be constructed of biocompatible materials, including but not limited to HydroThane™, polymeric materials, such as polyethylene, polypropylene, polyvinyl chloride, polytetrafluoroethylene, including expanded polytetrafluoroethylene (ePTFE), fluorinated ethylene propylene, fluorinated ethylene propylene, polyvinyl acetate, polystyrene, poly(ethylene terephthalate), naphthalene, dicarboxylate derivatives, such as polyethylene naphthalate, polybutylene naphthalate, polytrimethylene naphthalate and trimethylenediol naphthalate, polyurethane, polyurea, silicone rubbers, polyam ides, polyimides, polycarbonates, polyaldehydes, polyether ether ketone, natural rubbers, polyester copolymers, styrene-butadiene copolymers, polyethers, such as fully or partially halogenated polyethers, and copolymers and combinations thereof. Filler material 44 may further include one or more of metals, carbon fibers, glass fibers, and ceramics. Filler material 44 may coat an inner surface 46 of sheath 24, or it may coat an outer surface 48 of sheath 24. Alternatively, main body portion 30 of sheath 24 may be embedded within filler material 44. In some arrangements, at least a portion of sheath 24 may be dipped, coated, sprayed, or otherwise filled with filler material 44.

As shown in FIGS. 1 and 2, filler material 44 may extend along main body portion 30 of sheath 24 and terminate proximally of distal portion 32 of sheath 24. That is, while openings 28 of sheath 24 contained within body portion 30 are filled with filler material 44, openings 28 contained within distal portion 32 of sheath 24 are left unfilled with filler material 44 (e.g., empty or free from). As such, one or more medical devices 20 may be passed through openings 28 of distal portion 32 of sheath 24, as will be described in further detail below. Alternatively, filler material 44 may extend along main body portion 30 of sheath 24 and along distal portion 32 of sheath 24. As such, all openings 28 contained within sheath 24 may be filled with filler material 44. In such an arrangement, a distal end of sheath 24 may be spot welded to exterior surface 42 of body 36 such that one or more medical devices 20 may be passed through a distal—most end of sheath 24, between adjacent spot welds, rather than through openings 28 of distal portion 32 of sheath 24.

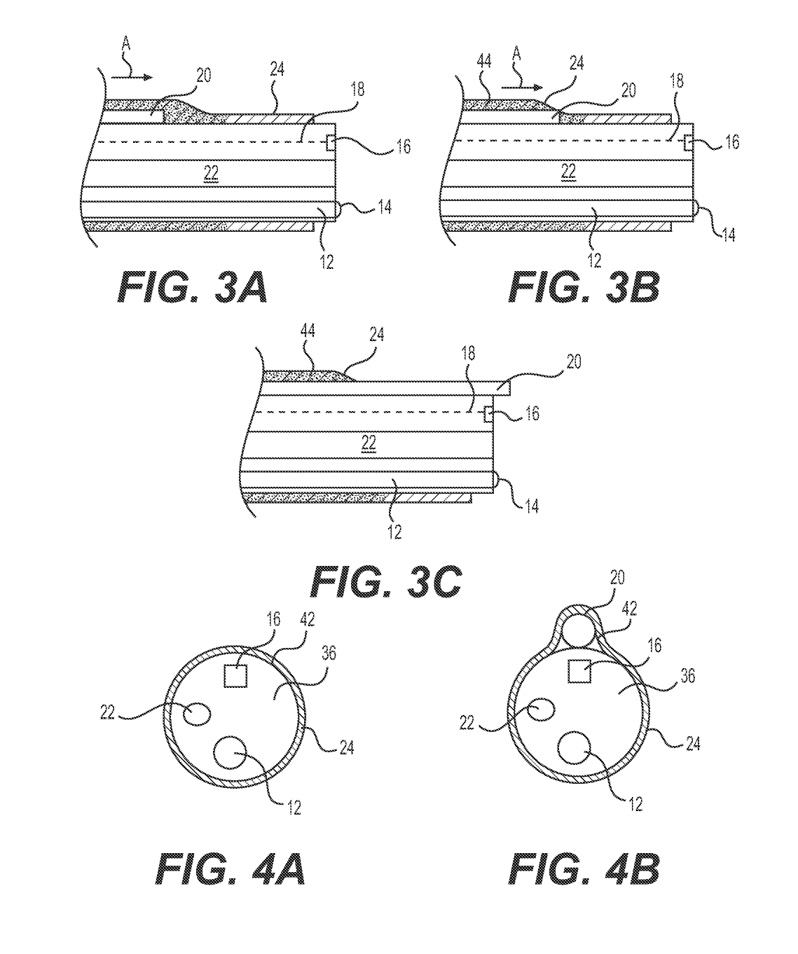

In use, as shown in FIGS. 3A-3C, a medical device 20 may be delivered into a subject's body via insertion device 10. As noted above, medical devices 20 may comprise one or more of guidewires, biopsy forceps, graspers, baskets, snares, probes, scissors, retrieval devices, laser fibers, and/or other tools configured for insertion into a subject's body. For example, as shown in FIG. 3A, a user may insert medical device 20 between sheath 24 and body 36 of insertion device 10. As medical device 20 is advanced distally in the direction A, as shown in FIG. 3B, sheath 24 expands to provide sufficient space for medical device 20 to pass. Due to the lubricious material or low coefficient of friction of filler material 44, medical device 20 may be urged through the entire length of sheath 24. Once a distal end of medical device 20 is advanced distally of filler material 44, medical device 20 may be free to pass through openings 28 (FIG. 2) of distal portion 32 of sheath 24, as shown in FIG. 3C. As such, medical device 20 may extend distally of sheath 24 such that medical tool 20 may be actuated to perform a treatment or procedure (e.g., excise tissue, capture tissue, ablate tissue, etc.).

Sheath 24 may facilitate low profile insertion of medical device 20 into a subject's body. For example, as shown in FIG. 4A, prior to insertion of medical device 20 between exterior surface 42 of body 36 and sheath 24, sheath 24 may fit snuggly about body 36, e.g., in full contact with body 36. Upon insertion of medical device 20 between exterior surface 42 of body 36 and sheath 24 as shown in FIG. 4B, sheath 24 expands only so much as necessary so as to pass medical device 20. That is, due to the expandable mesh of sheath 24 and filler material 44, an outermost profile of insertion device 10 is reduced. Additionally, it is understood that while only a single medical device 20 is depicted in FIGS. 3A-3C, 4A, and 4B, any number of medical devices may be passed between the exterior surface 42 and sheath 24. Additionally, while medical device 20 is depicted as extending along an upper or top surface of insertion device 10, it is understood that medical device 20 may be inserted along any circumferential position about exterior surface 42 of body 36. Accordingly, sheath 24 may facilitate low profile insertion of one or more medical devices 20 into a subject's body.

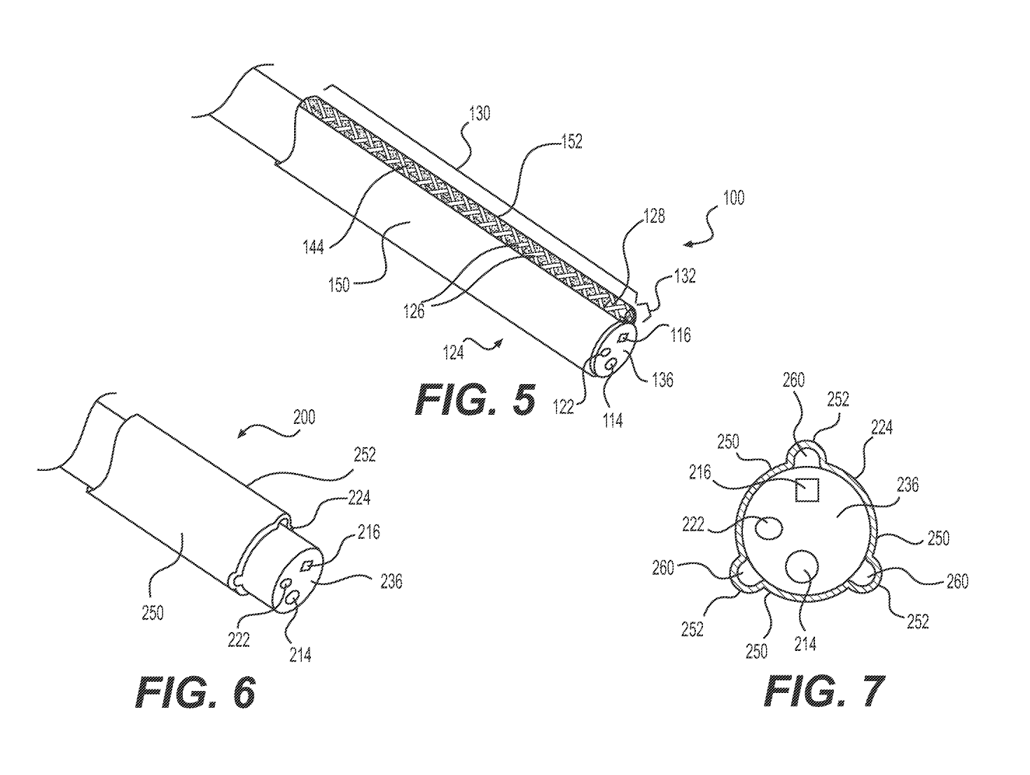

FIG. 5 illustrates a portion of an exemplary insertion device 100, according to an alternative arrangement. Insertion device 100 may be similar to insertion device 10. Accordingly, insertion device 100 may include a handle (not shown) and a longitudinally-extending body 136 extending distally of the handle. Similar to insertion device 10, insertion device 100 may include an illumination unit including, for example, a lens 114 aligned with a fiber optic light guide (not shown in FIG. 5). Insertion device 100 may also include an imaging unit 116, one or more irrigation/aspiration ports (not shown) for communicating fluid therethrough, one or more steering wires (e.g., Bowden cables) (not shown), and one or more working channels 122 configured to deliver one or more medical devices 20 therethrough.

Additionally, insertion device 100 may include a sheath 124. Sheath 124 may include one or more non-mesh portions 150 and one or more mesh portions 152, as shown in FIG. 5. Mesh portion(s) 152 and non-mesh portion(s) 150 may be monolithically (e.g., of a single one-piece construction) or may be separate components coupled to one another to form sheath 124. Similar to sheath 24 of FIG. 1, sheath 124 may be sized to be in full contact along body 136 absent an expanding or stretching force on mesh portions 152. Also, as shown in FIG. 5, mesh portion(s) 152 may extend axially along an entire length (e.g., from the proximal-most end to the distal-most end) of sheath 124 in a straight line. Non-mesh portion(s) 150 may be comprised of any appropriate material configured to surround body 136. Such materials may include, for example, nylon, polyurethane, urethanes, polyester, PTFE, PET, PEBA, EVA, or any other biocompatible material. Non-mesh portion(s) 150 may be less expandable and/or stretchable than mesh portion(s) 152. Additionally, similar to sheath 24, mesh portion(s) 152 may comprise a plurality of filaments 126 woven together. Similar to sheath 24 of FIG. 2, filaments 126 may be interconnected together to define a plurality of openings 128 between adjacent filaments 126 and may be woven together to form a dense, sparse, and/or varying density mesh as described above. Filaments 126 may comprise flexible wires and/or polymeric fibers. However, it is understood that the disclosure is not limited to woven filaments 126, but rather, includes other fabrications such as, for example, knitting, braiding, crocheting, welding, suturing, tying, twisting, or other such methods of producing interconnected mesh portion(s) 152. Exemplary materials for filaments 126 may include, but are not limited to, stainless steel, nylon, polyurethane, urethanes, polyester, Nitinol, PTFE, PET, PEBA, EVA, or any other biocompatible material. In an exemplary aspect, mesh portion(s) 152 may comprise a co-braid. In other words, mesh portion(s) 152 may comprise filaments 126 of two or more materials. In one example, mesh portion(s) 152 may comprise a co-braid of PTFE and Nitinol. While only a single mesh portion 152 is depicted, it is understood any number of appropriate mesh portions 152 may be used. Accordingly, multiple medical devices 20 may be delivered into a subject's body, each medical device 20 through one mesh portion 152. Additionally, it is understood that the one or more mesh portion(s) may be positioned at any appropriate circumferential position about body 136. Additionally, if multiple mesh portion(s) 152 are used, it is understood that the mesh portions 152 may be either symmetrically or asymmetrically spaced.

Similar to sheath 24, sheath 124 may be welded, glued, or otherwise joined to an exterior surface of body 136. In one exemplary aspect, sheath 124 may be tack welded or otherwise coupled to one or more portions of body 136 in any appropriate configuration. In one example, an entire circumferential surface of sheath 124 at a first axial location may be coupled to body 136. That is, a 360° weld may be formed between sheath 124 and an exterior surface at the first axial location. In other arrangements, a tack weld of less than 360° may be formed between sheath 124 and body 136. For example, one or more radial arcs of an entire circumferential surface of sheath 124 at a first axial location may be tack welded to an exterior surface of body 136. That is, for example, every other 30° of an entire circumferential surface of sheath 124 at a first axial location may be tack welded to an exterior surface of body 136. In other arrangements, every other 10-180° of an entire circumferential surface of sheath 124 at a first axial location may be tack welded to body 136. While symmetric coupling (e.g., every other 30°) has been described, it is to be understood that asymmetric coupling (e.g., a first arc of 30° and a space of less or more than 30°) is envisioned and contemplated by this disclosure. In other arrangements, one or more locations along an entire circumferential surface of sheath 124 at a first axial location may be spot welded to body 136. Such spot welds may be positioned at any desirable location about the circumferential surface of sheath 124 at a first axial location. In some arrangements, the sheath 124 may welded or otherwise coupled to body 136 along non-mesh portions 150 and not coupled to body 136 along mesh portions 152.

As shown in FIG. 5, one or more portions of mesh-portion(s) 152 may include a continuous filler material 144. Alternatively, the sheath 124 may be covered with a discontinuous filler material 144 along one or more portions of mesh-portion(s) 152. Such filler materials 144 may be comprised of a lubricious or low coefficient of friction material configured to facilitate passage of one or more medical devices 20 there along, as will be described in further detail below. Accordingly, the filler material 144 may be constructed of biocompatible materials, including but not limited to polymeric materials, such as polyethylene, polypropylene, polyvinyl chloride, polytetrafluoroethylene, including expanded polytetrafluoroethylene (ePTFE), fluorinated ethylene propylene, fluorinated ethylene propylene, polyvinyl acetate, polystyrene, poly(ethylene terephthalate), naphthalene, dicarboxylate derivatives, such as polyethylene naphthalate, polybutylene naphthalate, polytrimethylene naphthalate and trimethylenediol naphthalate, polyurethane, polyurea, silicone rubbers, polyam ides, polyimides, polycarbonates, polyaldehydes, polyether ether ketone, natural rubbers, polyester copolymers, styrene-butadiene copolymers, polyethers, such as fully or partially halogenated polyethers, and copolymers and combinations thereof. Filler material 144 may further include one or more of metals, carbon fibers, glass fibers, and ceramics.

As shown in 5, filler material 144 may extend along a main body portion 130 of mesh portion(s) 152 and terminate proximally of a distal portion 132 of mesh portion(s) 152. That is, while openings 128 of main body portion 130 of mesh portion(s) 152 are filled with filler material 144, openings 128 contained within distal portion 132 of main body portion 130 of mesh portion(s) 152 are unfilled with filler material 144. As such, one or more medical devices 20 may be passed through openings 128 of distal portion 132 of sheath 124, as will be described in further detail below. Alternatively, filler material 144 may extend along main body portion 130 and along distal portion 132 of sheath 124. As such, all openings 128 contained within sheath 124 may be filled with filler material 144. In such an arrangement, a distal end of sheath 124 may be spot welded to an exterior surface of body 136 such that one or more medical devices 20 may be passed through a distal-most end of sheath 124 rather than through openings 128 of distal portion 132 of mesh portion(s) 152.

In use, similar to FIGS. 1-4B, a medical device 20 may be delivered into a subject's body via insertion device 100. For example, a user may insert medical device 20 between sheath 124 and body 136 of insertion device 10. As noted above, medical devices 20 may comprise one or more of guidewires, biopsy forceps, graspers, baskets, snares, probes, scissors, retrieval devices, laser fibers, and/or other tools configured for insertion into a subjects body. Specifically, a user may insert one or more medical devices 20 between mesh portion(s) 152 and body 136. As medical device 20 is advanced distally, mesh portion(s) expand to provide sufficient space for medical device 20 to pass. Due to the lubricious material or low coefficient of friction of filler material 144, medical device 20 may urged through the entire length of sheath 124 along body 136. Once a distal end of medical device 20 is advanced distally of filler material 144, medical device 20 may be free to pass through openings 128 of distal portion 132 mesh portion(s) 152. As such, medical device 20 may extend distally of sheath 124 such that medical device 20 may be actuated to perform a treatment or procedure (e.g., excise tissue, capture tissue, ablate tissue, etc.).

Sheath 124 may facilitate low profile insertion of medical device 20 into a subject's body. For example, prior to insertion of medical device 20 between mesh portion(s) 152 of sheath 124 and body 136, sheath 124 may fit snuggly about body 136, e.g., in full contact with body 136. Upon insertion of medical device 20 between body 36 and mesh portion(s) 152, sheath 124 expands only so much as necessary so as to pass medical device 20. That is, due to the expandable mesh of sheath 124 and filler material 144, an outermost profile of insertion device 100 is reduced. Accordingly, sheath 124 may facilitate low profile insertion of one or more medical devices 20 into a subject's body.

FIG. 6 illustrates a portion of an exemplary insertion device 200, according to an alternative arrangement. Insertion device 200 may be similar to insertion devices 10 and 100. Accordingly, insertion device 200 may include a handle (not shown) and a longitudinally-extending body 236 extending distally of the handle. Similar to insertion device 10 and 100, insertion device 200 may include an illumination unit including, for example, a lens 214 aligned with a fiber optic light guide (not shown in FIG. 6). Insertion device 200 may also include an imaging unit 216, one or more irrigation/aspiration ports (not shown) for communicating fluid therethrough, one or more steering wires (e.g., Bowden cables) (not shown), and one or more working channels 222 configured to deliver one or more medical devices 20 therethrough.

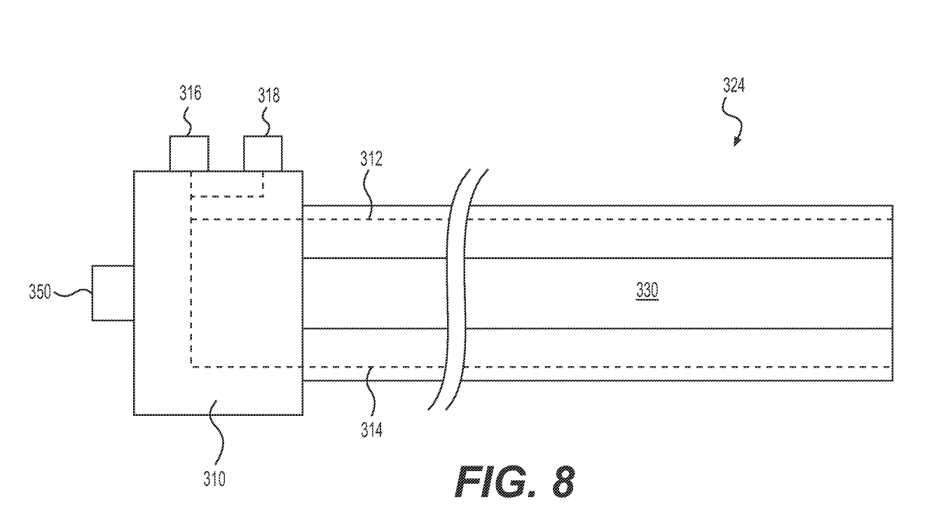

Additionally, insertion device 200 may include a sheath 224. Sheath 224 may have a preformed shape including one or more main portion(s) 250 and one or more channel(s) 252 extending along sheath 224. As shown in FIG. 7, main portion(s) 250 of sheath 224 may be configured to fit snuggly along body 236 (e.g., in full contact with body 236) of insertion device 200 while channel(s) 252 may extend radially outward of main portion(s) 250 so as to provide space 260 configured to pass a medical device 20 therethrough. Sheath 224 may be comprised of a lubricious or low coefficient of friction material configured to facilitate passage of one or more medical devices 20 through channels 252, as will be described in further detail below. Accordingly, the sheath 224 may be constructed of biocompatible materials, including but not limited to polymeric materials, such as polyethylene, polypropylene, polyvinyl chloride, polytetrafluoroethylene, including expanded polytetrafluoroethylene (ePTFE), fluorinated ethylene propylene, fluorinated ethylene propylene, polyvinyl acetate, polystyrene, poly(ethylene terephthalate), naphthalene, dicarboxylate derivatives, such as polyethylene naphthalate, polybutylene naphthalate, polytrimethylene naphthalate and trimethylenediol naphthalate, polyurethane, polyurea, silicone rubbers, polyam ides, polyimides, polycarbonates, polyaldehydes, polyether ether ketone, natural rubbers, polyester copolymers, styrene-butadiene copolymers, polyethers, such as fully or partially halogenated polyethers, and copolymers and combinations thereof. Sheath 224 may further comprise one or more of metals, carbon fibers, glass fibers, and ceramics.

In use, a medical device 20 may be delivered into a subject's body via insertion device 200. For example, a user may insert medical device 20 between sheath 224 and body 236 of insertion device 200 via one or more channel(s) 252. Once a distal end of medical device 20 is advanced distally of channel 252, medical device 20 may be actuated to perform a treatment or procedure (e.g., excise tissue, capture tissue, ablate tissue, etc.). Similar to sheaths 24 and 124, sheath 224 may facilitate low profile insertion of medical device 20 into a subject's body. That is, by virtue of sheath 224, one or more medical devices 20 may be delivered in close proximity of body 236.

While each of body 36, 136, and 236 are illustrated as containing a circular cross-section, it is understood that the disclosure is not so limited. Rather, any of bodies 36, 136, and 236 may have any cross-sectional shape such as, for example, circular, ovular, irregular, and/or any other cross-sectional shape appropriate for insertion within anatomical lumens of the subject's body, and/or combinations thereof.

FIG. 8 depicts an exemplary sheath 324 including optional additional features of sheaths 24, 124, and 224. As shown in FIG. 8, sheath 324 defines a central passage 330 and extends distally of a handle 310. Passage 330 may be configured to receive a body portion 36, 136, 236 of an insertion device 10, 100, 200 therein. In another arrangement, however, passage 330 may be configured to receive medical device 20 therein. In such an arrangement, medical device 20 may be independently steered (e.g., articulated) and/or locked in position as will be described in further detail below. Accordingly, sheath 324 may enable additional articulation movement of medical device 20 independent of insertion device 10, 100, 200. Although only a single passage 330 has been depicted, it is understood that any appropriate number of passages 330 may be disposed through sheath 324 without departing from the scope of this disclosure. Body portion 36, 136, 236 and/or medical device 20 may be inserted into passage 330 though any appropriate port (not shown) on handle 310.

Handle 310 may include one or more actuators 316 configured to facilitate deflection of sheath 324. For example, actuator 316 may be operably coupled one or more articulation wires 312, 314. Manipulation of actuator 316 may deflect, bend, or otherwise adjust the positioning of a distal portion of sheath 324. For example, distal (e.g., forward) movement of actuator 316 may cause the distal portion of sheath 324 to move in a first direction (e.g., downward) along a plane by placing articulation wire 314 in tension. Additionally, proximal (e.g., backward) movement of actuator 316 may cause the distal portion of sheath 324 to move in a second direction (e.g., upward) along the same plane by placing articulation wire 312 in tension. Accordingly, sheath 324 may be steered so as to transition between a first configuration and a second configuration, different than the first configuration. It is understood that these directions may be reversed without departing from the scope of this disclosure. In such a manner, if a medical professional determines a need or desire to alter the positioning of the distal portion of sheath 324, he/she may do so via actuator 316. While actuator 316 is schematically illustrated, it is understood that actuator 316 may comprise a joystick, slider, a push button, a two-way rocker, and/or other such arrangements. Additionally, while only two-way deflection is described, it is understood that greater of fewer degrees of movement may be achieved. For example, in some arrangements, four-way deflection (e.g., up, down, right, and left) may be achieved via an alternative actuator 316, such as, for example, a joystick. Additionally, it is understood that in some exemplary aspects, articulation wires 312, 314 and actuator 316 may eliminate the need for a similar articulation system of body 36, 136, 236 and as such, body 36, 136, 236 may have a reduced profile.

Sheath 324 may further include a locking mechanism. For example, as shown in FIG. 8, handle 310 may include at least one lock 318. Lock 318 may be configured to maintain a desired arrangement of sheath 324. For example, manipulation of lock 318 may retain articulation wires 312, 314 in place such that sheath 324 is held in position. Accordingly, lock 318 may prevent inadvertent movement of sheath 324. Additionally, lock 318 may maintain a distal end of sheath 324 in place such that body 36, 136, 236 or medical device 20 may be consistently delivered into a particular location within a subject's body through passage 330.

Additionally or alternatively, sheath 324 may include an inner lining (not shown) positioned within passage 330. The inner lining may be comprised of any readily expansible material. For example, in some arrangements, the inner lining may be comprised of an inflatable balloon member which may be selectively inflated so as to expand and or dilate sheath 324. For example, the inner lining may be fluidly coupled to a source of inflation fluid (e.g., gas or liquid). Upon expansion of the inner lining, sheath 324 may radially expand. Additionally or alternatively, handle 310 may include a fluid coupler such as a luer 350. Luer 350 may be configured to communicate fluid to and/or from sheath 324. For example, luer may couple sheath 324 to a source of vacuum pressure such that sheath 324 may be used to suction material therethrough.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the disclosure is not to be considered as limited by the foregoing description.

We claim:

1. A medical device, comprising:
a longitudinally extending body defining an exterior surface and configured for into a body of a patient; and
a sheath coupled to and surrounding at least a portion of the longitudinally extending body;
wherein the sheath comprises a selectively expandable mesh of interconnected filaments defining openings between adjacent filaments of the mesh, the mesh configured to expand to pass a medical device along the longitudinally extending body, between the mesh and the exterior surface of the longitudinally extending body, and through an opening between adjacent filaments of the mesh.

2. The medical device of claim 1, wherein the mesh of interconnected filaments surrounds an entire circumferential surface of the longitudinally extending body.

3. The medical device of claim 1, wherein the sheath further comprises one or more non-mesh portions.

4. The medical device of claim 1, wherein the filaments comprise at least one of flexible wires or polymeric fibers.

5. The medical device of claim 1, wherein the mesh of interconnected filaments comprises filaments of two or more materials.

6. The medical device of claim 5, wherein the two or more materials include at least PTFE and Nitinol.

7. The medical device of claim 1, further comprising a filler material extending along at least a portion of the sheath.

8. The medical device of claim 7, wherein the filler material comprises a polymeric material.

9. The medical device of claim 8, wherein the filler material fills in openings between adjacent filaments along the portion of the sheath.

10. The medical device of claim 7, wherein the filler material includes a lubricious coating.

11. The medical device of claim 7, wherein the filler material terminates proximally of a distal-most end of the sheath.

12. The medical device of claim 1, wherein a distal end of the sheath is fixed to the longitudinally extending body.

13. A method, comprising:
delivering a longitudinally extending body into an anatomical opening of a patient, the body defining an exterior surface, at least a portion of the exterior surface being surrounded by a sheath;
advancing a medical device along the exterior surface so as to expand the sheath radially outward of the exterior surface, wherein the sheath comprises a selectively expandable mesh of interconnected filaments defining openings between adjacent filaments of the mesh; and
extending the medical device through an opening between adjacent filaments of the mesh.

14. The method of claim 13, further comprising:
actuating the medical device to perform a treatment.

15. The method of claim 13, wherein the mesh of interconnected filaments surrounds an entire circumferential surface of the body.

16. The method of claim 13, wherein the sheath further comprises one or more non-mesh portions.

17. The method of claim 13, wherein the sheath further comprises a filler material extending along at least a portion of the sheath.

18. A medical device, comprising:
a longitudinally extending body defining an exterior surface and configured for insertion into a body of a patient;
a sheath surrounding at least a portion of the longitudinally extending body; and
a filler material extending along at least a portion of the sheath;
wherein the sheath comprises a selectively expandable mesh of interconnected filaments defining openings between adjacent filaments of the mesh, the mesh of interconnected filaments surrounding an entire circumferential surface of the longitudinally extending body and configured to expand to pass a medical device along the longitudinally extending body, between the mesh and the exterior surface, and through an opening between adjacent filaments of the mesh.

19. The medical device of claim 18, wherein the filler material fills in openings between adjacent filaments along the portion of the mesh.

20. The medical device of claim 18, wherein the mesh of interconnected filaments comprises filaments of two or more materials, wherein the two or more materials include at least PTFE and Nitinol.
